(19) United States
(12) Patent Application Publication
(10) Pub. No.: US 2022/0371060 A1
(43) Pub. Date: Nov. 24, 2022

(54) Adjustable Cleaning and Sanitizing Apparatus

(71) Applicant: Kent Brown, Smyrna, DE (US)

(72) Inventor: Kent Brown, Smyrna, DE (US)

(21) Appl. No.: 17/751,334

(22) Filed: May 23, 2022

Related U.S. Application Data
(60) Provisional application No. 63/191,727, filed on May 21, 2021.

(51) Int. Cl. B08B 3/02; B08B 13/00; A61L 2/26; A61L 2/18; B05B 13/02

(52) U.S. Cl. CPC B08B 3/02; B08B 3/024; B08B 13/00; A61L 2/26; A61L 2/18; B05B 13/0278; B08B 2203/0211; B08B 2203/0264; A61L 2202/15; F21S 8/065

(57) ABSTRACT

A flexible cleaning apparatus comprising a plurality of extendible tubes disposed in fluidic communication with each other and is arranged in a three-dimensional geometry that defines an internal cleaning region. The flexible cleaning apparatus has a cleaning fluid interface configured to provide cleaning solution to the plurality of extendible tubes. The flexible cleaning apparatus has a plurality of nozzles disposed along the plurality of extendible tubes and is configured to transit the cleaning solution from the plurality of extendible tubes to the internal cleaning region. The plurality of extendible tubes are each configured to adjust between a first length and a second length to adjust a volume of the internal cleaning region.

### CROSS-REFERENCE TO RELATED APPLICATION

This application claims priority to U.S. Patent Application No. 63/191,727, titled “Adjustable Cleaning and Sanitizing Apparatus,” and filed on May 21, 2021. The entire application is incorporated herein by reference in its entirety.

### FIELD OF THE INVENTION

This disclosure generally relates to cleaning and sanitizing suspended objects. Specifically, the invention relates to an adjustable cleaning and sanitizing apparatus and related methods of use.

### BACKGROUND

Suspended fixtures, such as chandeliers, typically hang from a ceiling for decorative or lighting purposes. Over time, dust, cobwebs and other filth can accumulate on such suspended devices that may diminish their decorative appearance and act as a reservoir for allergens.

In order to combat these problems, it is important to periodically clean or sanitize such suspended fixtures. Typically, cleaning must be done in place using dusting cloths and/or cleaning solutions applied by a spray bottle or aerosol. Alternatively, if greater care is needed, the chandelier may be removed from a ceiling in parts or in whole so that cleaning and/or dusting can be done on a flat surface to reduce the risk of damage to the chandelier.

Unfortunately, there are deficiencies to the above-described conventional approaches to cleaning and sanitizing a suspended fixture. For example, with a cleaning in place approach, the user risks damaging the chandelier by dislodging decorative components that may fall to the floor and break. Alternatively, if removal from the ceiling is utilized, a chandelier is still at risk of breakage during removal and/or reinstallation. Additionally, the removal, cleaning, and reinstallation process can be time consuming and labor intensive.

There exists a need to improve upon the above-identified conventional approaches to cleaning and sanitizing a suspended fixture. The following disclosure includes systems and methods for cleaning a suspended fixture without the need for removing the fixture from its installation location.

### SUMMARY OF THE INVENTION

The present invention is directed towards an improved cleaning and sanitizing apparatus and related methods of use. Some embodiments of the present invention comprise an adjustable cleaning and sanitizing device capable of extending or reducing to a size sufficient to surround a suspended fixture and applying a cleaning and sanitizing solution to the suspended fixture in an efficient and thorough manner without the need for removal of the fixture from its installed location.

One embodiment is directed to a flexible cleaning apparatus. The flexible cleaning apparatus has a plurality of extendible tubes disposed in fluidic communication with each other and is arranged in a three-dimensional geometry that defines an internal cleaning region. The flexible cleaning apparatus has a cleaning fluid interface configured to provide cleaning solution to the plurality of extendible tubes. The flexible cleaning apparatus has a plurality of nozzles disposed along the plurality of extendible tubes and is configured to transit the cleaning solution from the plurality of extendible tubes to the internal cleaning region. The plurality of extendible tubes are each configured to adjust between a first length and a second length to adjust a volume of the internal cleaning region.

### BRIEF DESCRIPTION OF THE DRAWINGS

The following drawings are intended to serve as exemplary embodiments of the features disclosed in the present disclosure.

FIG. 1 is a perspective view of a cleaning and sanitizing system having extendable tubes, a plurality of nozzles, and an interface for receiving a supply of cleaning and sanitizing solution;

FIG. 2 is an additional perspective view of a cleaning and sanitizing system, further comprising a plurality of wheels; and

FIG. 3 is a cross-sectional view of one tube of the plurality of extendible tubes.

### DETAILED DESCRIPTION OF DRAWINGS

The description of illustrative embodiments according to principles of several illustrative embodiments is intended to be read in connection with the accompanying drawings, which are to be considered part of the entire written description. In the description of embodiments disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits are illustrated by reference to certain exemplified embodiments and may not apply to all embodiments.

Accordingly, the invention expressly should not be limited to such exemplary embodiments illustrating some possible non-limiting combination of features that may exist alone or in other combinations of features; the scope of the claimed invention being defined by the claims appended hereto.

This disclosure describes the best mode or modes of practicing the invention as presently contemplated. This description is not intended to be understood in a limiting sense, but provides an example of the invention presented solely for illustrative purposes by reference to the accompanying drawings to advise one of ordinary skill in the art of the advantages and construction of the invention. In the various views of the drawings, like reference characters designate like or similar parts.

The various embodiments described herein describe systems and methods relating to a cleaning and sanitizing suspended objects. Embodiments of the present invention may provide a geometrically shaped three-dimensional arrangement of hollow tubes that can be extended or retracted in length and have a plurality of nozzles capable of directionally applying a cleaning solution from within the hollow tubes to an object disposed within the three-dimensional arrangement. Accordingly, the object that may be of a variety of different sizes may be cleaned efficiently and thoroughly while minimizing the risk of damage to the object and without the need to remove the object from its installed location.

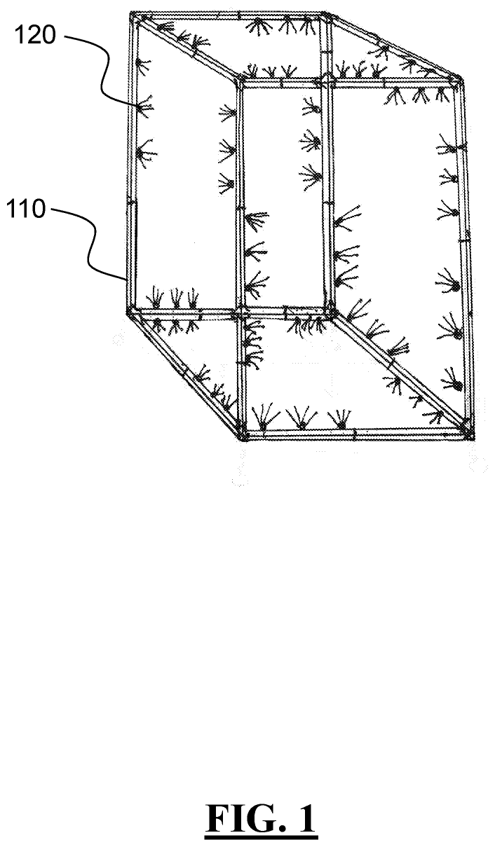

FIG. 1 shows a plurality of extendable tubes 110 arranged in a cubic shape of which at least one of the tubes includes a nozzle 120. While a cubic arrangement is depicted in FIG. 1, other arrangements are possible. For example, the extendible tubes can be arranged as a pyramid, a cylinder, a sphere, or any other three-dimensional shape that will accommodate an interior cleaning region. Since the extendible tubes 110 are capable of changing length, the volume and dimensions of the interior cleaning region are adjustable. As seen in FIG. 1, where the adjustable tubes 110 are arranged in a cubic shape, the volume of the interior cleaning region can be changed dramatically with a relatively small change in the length of the extendable tubes. For example, if the length of the extendible tubes 110 are each increased from a length of one foot to two feet, the volume of the internal cleaning region would increase from one cubic foot to eight cubic feet. While the arrangement depicted in FIG. 1 shows all tubes 110 of the cubic shape being extendible, this need not be the case. If adjustment is only needed in one dimension, only four of the twelve tubes 110 need be extendible. Alternatively, if adjustment is needed only in two dimensions, only eight of the tubes 110 need be extendible. Finally, if adjustment is needed in three dimensions, all twelve of the tubes 110 may be extendible.

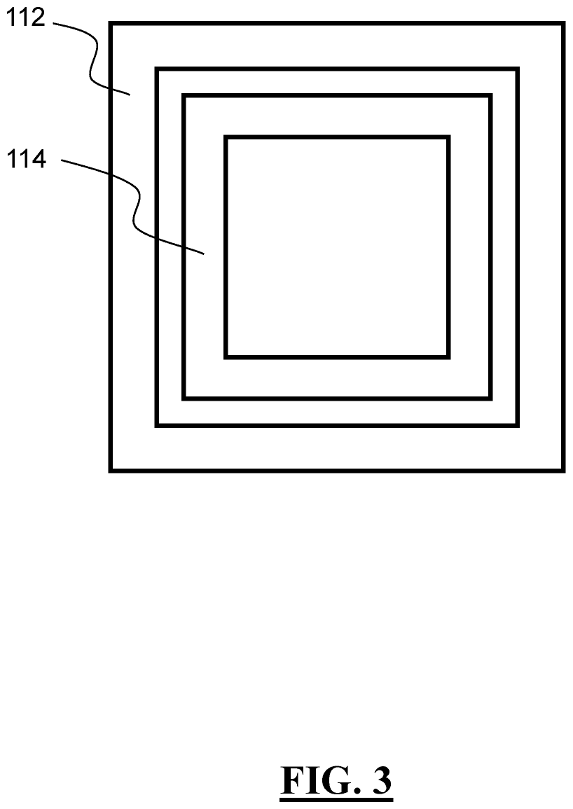

As shown in FIG. 1 a change in length of the extendible tubes 110 may be accomplished by using telescopic elements. With reference to FIG. 3, a cross-sectional view of an extendable tube 110 using telescopic elements is shown. Although FIG. 3 shows an exemplary extendible tube 110 that is square in shape, it is contemplated that the tube 110 may be made in according to any geometric shape. As shown in FIG. 3, such telescopic elements could include at least two tubular elements of differing diameters such that an outer diameter of a thinner tube 114 can pass through an inner diameter of a wider tube 112. The number of telescopic elements per adjustable tube may be chosen based on the desired range of size adjustments for the cleaning and sanitizing device. For example, an extendible tube with two telescopic elements can double in length; an extendible tube with three telescopic elements can triple in length; and so on. While FIG. 1 depicts the change in length of the extendible tubes via telescoping, other arrangements are possible to achieve similar adjustable lengths. For example, accordion regions (similar to the bendable region of a bendable drinking straw) may be utilized in part or in all of an extendable tube. The use of accordion regions not only allow for a change in length, but also allow for a change in shape of the overall three-dimensional arrangement of the extendible tubes by allowing those tubes to bend. This would advantageously allow greater flexibility in the shaping of the cleaning and sanitizing device to accommodate particular objects to be cleaned/sanitized. In addition to telescoping or using accordion regions, screw connections or any other suitable structure for changing the length of a tube may be used to achieve the size changing purposes of the invention. In some embodiments, the telescoping elements may further include locking wheels to prevent the apparatus from shifting or moving during operation.

As shown in FIG. 1, a plurality of nozzles 120 are arranged along the extendible tubes 110 for allowing cleaning and sanitizing solution to be applied to an object located within the internal cleaning region. The nozzles 120 may be static, such as orifices in the wall of the extendible tube 110. Alternatively, the nozzles 120 may be adjustable with respect to an angular direction. This may be accomplished, for example, by a ball and socket connection similar to a shower head. Additionally, the nozzles 120 may be adjustable with respect to a location along the extendible tube 110. This can be accomplished, for example, by having a nozzles capable of sliding along a track within the extendible tube. Additionally, the nozzles 120 may be adjustable with respect to nozzle diameter. This can be accomplished, for example, by implementing variable diameter nozzles, wherein the opening of the nozzles may be adjusted with respect to diameter. This may would advantageously allow the user to increase or decrease the pressure of the solution ejected from the nozzles. Additionally, the nozzles 120 may be adjustable with respect to spray pattern. Accordingly, this may be accomplished by using nozzles configured to adjust spray pattern. The spray pattern can be adjusted for the exact dimensions of the object to be cleaned, ensuring that the object will be precisely cleaned with minimal overspray. The spray pattern may be adjusted anywhere from 0 to 360 degrees, depending on the desirable spray pattern used for an object. For example, a chandelier with larger crystals may be more effectively cleaned with the use of nozzles configured with a wider spray pattern while a chandelier with smaller crystals may be more effectively cleaned with the use of nozzles configured for a narrower spray pattern. Additionally, the nozzles 120 may be adjustable between an open-position and a closed-position. This may be accomplished by using a valve, for example a ball valve, or any other type of suitable valve. This would advantageously allow the user to adjust how many of the nozzles 120 are configured to emit cleaning solution to provide even greater precision when cleaning and/or sanitizing an object. The nozzles 120 can be disposed along some or all of the extendible tubes allowing for up to 360-degree coverage of an object to be cleaned and sanitized.

In some embodiments, the nozzles 120 may also be configurated to oscillate. This would advantageously allow greater control over washing chandeliers and may be especially advantageous for washing chandeliers with a high number of delicate crystals. This may be accomplished by using a nozzle capable of rotating in a 0° spray pattern. Accordingly, the centrifugal force produces a strong impact and a spray pattern to advantageously increase scrubbing ability of the apparatus during the cleaning process. In one embodiment, the oscillating nozzle 120 may utilize low and high pressures in a chamber that causes the water stream to oscillate back and forth. In some embodiment, the nozzle 120 may be rotary nozzle configured to spin a zero-degree spray in a circle. Accordingly, the spinning water spray hits the surface area from multiple angles. Furthermore, the spray pattern may be adjusted by adjusting the distance between the nozzle and the surface being cleaned.

The extendible tubes may be made of a variety of different materials to accentuate certain advantages of the cleaning/sanitizing device. For example, the extendible tubes may be made of metals such as stainless steel or aluminum for strength and corrosion resistance. The extendible tubes may be made of plastics such as polyethylene or PVC for lightness of weight. The extendible tubes may be made of rubbers or silicone to allow for additional flexibility in shape.

In use, the cleaning and sanitizing device is adjusted to fit about the object, such as a chandelier, to be cleaned. The nozzles are arranged to point toward the object to be cleaned. Cleaning solution is then passed though the extendible tubes and into the nozzles which then cover the object in cleaning solution. Cleaning solution is supplied to the extendible tubes via a supply interface. The cleaning solution may be any fluid capable of cleaning the desired object. Such fluids may include water, detergent, alcohol, or any other chemical composition suitable for cleaning/sanitizing. One or more cleaning cycles may be desirable depending on the situation or object to be cleaned. For example, a single application of alcohol through the device may be sufficient to provide adequate cleaning and will evaporate on its own without additional steps. Alternatively multiple cleaning cycles may be utilized, such as an initial cycle with detergent and a subsequent rinsing cycle with water.

In some embodiments, the apparatus may be configured to connect to an external water source. In some embodiments, the apparatus may be configured to connect to a faucet. In some embodiments, the apparatus may be configured to recycle the water uses in a self contained system.

In some embodiments, the supply interface may be configured to pass air in alternative to cleaning solution. For example, hot air may be supplied to an object after the object has undergone a cleaning cycle in order to dry the object. It is contemplated that air may be transported throughout the device using the same passage in the supply interface as the passage used to transport cleaning solution. It is further contemplated that air may be supplied through a different passage than the passage used to supply cleaning solution in the supply interface.

In some embodiments, the present invention may include a water-impermeable curtain around the outside of the apparatus to contain any overspread and/or dirty cleaning solution. Embodiments may further include an impermeable tarp or other catch basin below the apparatus, wherein the impermeable tarp or catch basin may include a drain hole in order to collect and remove the used cleaning solution and overspray. Such an impermeable tarp or catch basin may provide the further advantage of preventing dislodged decorative components of the hanging fixture from fall to the ground and becoming damaged.

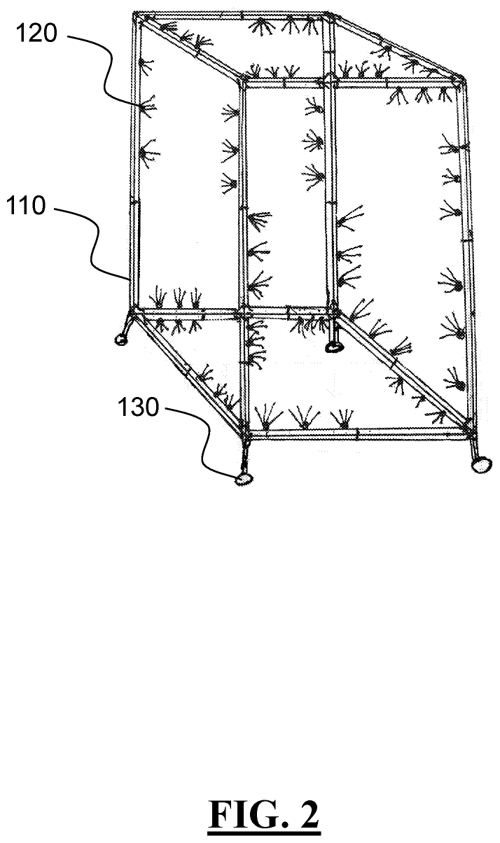

As shown in FIG. 2. in some embodiments, the present invention may include a plurality of wheels 130 disposed on the apparatus. In some embodiments, the plurality of wheels is mounted to extendible tubes which are telescopic in nature. While a four-wheel arrangement is depicted in FIG. 2, other arrangements comprising any number of wheels are possible. In one embodiment, the apparatus may include a total of four wheels 130 mounted on the bottom of the apparatus, wherein one set of two wheels 130 is mounted on the front side of bottom of the apparatus, and a second set of two wheels 130 is mounted on the backside of the bottom of the apparatus. Such a plurality of wheels may provide a further advantage of improving the maneuverability of the apparatus.

In some embodiments, the present invention may include miniature cameras mounted at strategic locations around the apparatus, such as at each of the sides of the apparatus, to allow for monitoring of the invention during operation. Furthermore, in some embodiments, such cameras may be operably coupled to a computer and may be configured to communicate with the computer. It is contemplated that the cameras may be connected to the computer with a wire or wirelessly. Furthermore, the computer may be connected to a peripheral screen to allow the user to view the operation of the apparatus as it is ongoing. In one embodiment, the cameras are connected wirelessly to a smart device, such as a smartphone, tablet, or any other suitable viewing device, and a user may be able to monitor the operation of the apparatus from the smartphone or other handheld device.

While various embodiments of the invention have been particularly shown and described, it will be understood by those skilled in the art that various changes in form and details may be made therein without departing from the spirit and scope of the invention as defined by the appended claims.

For example, while the cleaning and sanitizing device can be utilized for cleaning hard to reach objects such as chandeliers, other applications are possible. For example, the device can be used for watering plants, painting objects, applying pesticides, or any other application where the dispersion of a fluid is needed.

In another example, while the nozzles are described above as facing inward toward the cleaning/sanitizing region, the nozzles could alternatively face outward to apply fluids to an outside environment while avoiding contact with an internal region where, for example, a user may be located.

One having skill in the art will understand that certain combinations and/or sub-combinations of elements and features are of utility and may be employed without reference to other combinations and/or sub-combinations and are contemplated within the scope of the claims. Not all steps listed in the various figures need be carried out in the specific order described.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. Furthermore, the foregoing describes the invention in terms of embodiments foreseen by the inventor for which an enabling description was available, notwithstanding that insubstantial modifications of the invention, not presently foreseen, may nonetheless represent equivalents thereto.

What is claimed is:

1. A flexible cleaning apparatus, comprising:
a plurality of extendible tubes disposed in fluidic communication with each other, the plurality of extendible tubes being arranged in a three-dimensional geometry that defines an internal cleaning region,
wherein the plurality of extendible tubes are each configured to adjust between a first length and a second length to adjust a volume of the internal cleaning region;
a cleaning fluid interface configured to provide cleaning solution to the plurality of extendible tubes; and
a plurality of nozzles disposed along the plurality of extendible tubes, the plurality of nozzles being configured to transit the cleaning solution from the plurality of extendible tubes to the internal cleaning region.

2. The cleaning apparatus of claim 1, wherein the plurality of nozzles is configured to adjust with respect to the angular position of the nozzle.

3. The cleaning apparatus of claim 1, wherein the plurality of nozzles is configured to adjust with respect to nozzle diameter.

4. The cleaning apparatus of claim 1, wherein the plurality of nozzles is configured to adjust with respect to location of the nozzles on the extendible tubes.

5. The cleaning apparatus of claim 1, wherein the plurality of nozzles is configured to oscillate.

6. The cleaning apparatus of claim 1, wherein the plurality of nozzles is disposed in a static position on the plurality of extendible tubes.

7. The cleaning apparatus of claim 1, wherein the plurality of nozzles is configured to move along a rail system disposed on the plurality of extendible tubes.

8. The cleaning apparatus of claim 1, further comprising a plurality of wheels disposed on the extendible tubes.

9. The cleaning apparatus of claim 1, further comprising a water-impermeable tarp disposed around the apparatus configured for containing overspray during cleaning.

10. The cleaning apparatus of claim 1, further comprising a basin disposed below the apparatus configured for catching used cleaning solution.

11. The cleaning apparatus of claim 9, further comprising a drain hole configured to remove used cleaning solution caught by the basin.

12. The cleaning apparatus of claim 1, further comprising at least one camera, the at least one camera disposed on at least one of the extendible tubes of the plurality of extendible tubes, wherein the camera is configured to show the apparatus during operation.

13. The cleaning apparatus of claim 12, further comprising a viewing device configured to display a visual recording captured by the camera.

14. A method for cleaning an object, the method comprising the steps of
adjusting a cleaning apparatus to fit about an object, the apparatus comprising a plurality of extendible tubes, a plurality of nozzles, and a supply interface;
arranging the plurality of nozzles to point towards the object;
supplying the extendible tubes with a cleaning solution;
emitting the cleaning solution from the plurality of nozzles; and
covering the object with the cleaning solution.

15. The method of claim 14, further comprising adjusting the plurality of nozzles with respect to the location of the nozzles disposed on the plurality of extendible tubes.

16. The method of claim 14, further comprising adjusting the plurality of nozzles with respect to angular direction.

17. The method of claim 14, further comprising adjusting the plurality of nozzles with respect to nozzle diameter.

18. The method of claim 14, further comprising adjusting the plurality of nozzles with respect to spray pattern.

19. The method of claim 14, further comprising monitoring the apparatus with a camera while the apparatus is operating.
